(19) United States
(12) Patent Application Publication
(10) Pub. No.: US 2015/0147389 A1
(43) Pub. Date: May 28, 2015

(54) NOVEL ANTIOXIDANT FORMULATIONS

(71) Applicant: Phillip E. Schoenwetter, San Pedro, CA (US)

(72) Inventor: Phillip E. Schoenwetter, San Pedro, CA (US)

(21) Appl. No.: 14/550,859

(22) Filed: Nov. 21, 2014

Related U.S. Application Data
(60) Provisional application No. 61/907,559, filed on Nov. 22, 2013.

(51) Int. Cl. A61K 36/296; A61K 31/353; A61K 31/7048; A61K 31/05; A61K 31/375; A61K 31/385; A61K 31/015; A61K 31/198; A61K 31/355

(52) U.S. Cl. CPC A61K 36/296; A61K 31/198; A61K 31/353; A61K 31/7048; A61K 31/355; A61K 31/375; A61K 31/385; A61K 31/015; A61K 31/05
USPC 424/451; 424/725

(57) ABSTRACT

An antioxidant composition comprises one or more amino acids; a genistein alternative selected from the group consisting of genistein, aglycon genistine, hormone receptive modulators, phosphordiesterase (PDE) inhibitors of types 1, 2, 3 and 4, anti-neoplastic tyrosine kinase inhibitors, and combinations thereof; and an icariin alternative selected from the group consisting of icarosides 1 or 2, PDE-4 inhibitors, PDE-5 inhibitors, derivatives of the epimedium species including icarrocides I and II, and combinations thereof.

### CROSS REFERENCE TO RELATED APPLICATIONS

This application claims the benefit of U.S. Provisional Application No. 61/907,559 filed Nov. 22, 2013, which is incorporated herein by reference in its entirety.

### FIELD OF THE INVENTION

The present invention relates to novel formulations for mitigating oxidative stress and treating a variety of conditions and ailments; and more particularly to antioxidant compositions as well as mitigating adverse premature loss of gonad hormones as a result of spay and neutering dogs and cats.

### BACKGROUND OF THE INVENTION

Studies have shown that regulating multimodal metabolic pathways provides benefits including, but not limited to, free radical quenching, support and production of endogenous antioxidants, osteoblastic and neurogenerative stimulation, anti-inflammatory and anti-autoimmune functions, neuroprotection, and mitigation of the effect of ionizing radiation (UV/X-Ray/Gamma Radiation). Many of these benefits are produced from mechanisms other than mere free radical quenching and have been found to mediate up-regulation of certain genes.

### SUMMARY OF THE PREFERRED EMBODIMENTS

In accordance with a first aspect of the present invention there is provided an antioxidant composition comprising one or more amino acids, a genistein alternative and an icariin alternative.

In accordance with another aspect of the present invention there is provided a method for a mammal to mitigate oxidative stress with an antioxidant composition comprising one or more amino acids, a genistein alternative and an icariin alternative.

In accordance with yet another aspect of the present invention there is provided a method of treating conditions or ailments of a mammal with an antioxidant composition comprising one or more amino acids, a genistein alternative and an icariin alternative.

The invention, together with additional features and advantages thereof, may be best understood by reference to the following description.

### DETAILED DESCRIPTION OF THE PREFERRED EMBODIMENTS

The following description is illustrative and is not to be construed as limiting. Numerous specific details are described to provide a thorough understanding of the disclosure. However, in certain instances, well-known or conventional details are not described in order to avoid obscuring the description. References to one or an other embodiment in the present disclosure can be, but not necessarily are, references to the same embodiment; and, such references mean at least one of the embodiments.

Reference in this specification to “one embodiment” or “an embodiment” means that a particular feature, structure, or characteristic described in connection with the embodiment is included in at least one embodiment of the disclosure. Appearances of the phrase “in one embodiment” in various places in the specification do not necessarily refer to the same embodiment, nor are separate or alternative embodiments mutually exclusive of other embodiments. Moreover, various features are described which may be exhibited by some embodiments and not by others. Similarly, various requirements are described which may be requirements for some embodiments but not other embodiments.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure.

Consequently, alternative language and synonyms may be used for any one or more of the terms discussed herein. Nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any exemplified term. Likewise, the disclosure is not limited to various embodiments given in this specification.

Without intent to further limit the scope of the disclosure, examples of compositions, kits, methods and their related results according to the embodiments of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions, will control.

In a preferred embodiment of the present invention, the antioxidant composition may comprise one or more amino acids, preferably those that limit the production of glutathione, a genistein alternative selected from the group consisting of genistein, aglycon genistine, hormone receptive modulators, phosphordiesterase (PDE) inhibitors of types 1, 2, 3 and 4, anti-neoplastic tyrosine kinase inhibitors, selective estrogen Receptor modulator (SERM) type beta, and combinations thereof; and an icariin alternative selected from the group consisting of icarosides 1 or 2, PDE-4 inhibitors, PDE-5 inhibitors, derivatives of the epimedium species including icarrocides I and II, and combinations thereof. Preferably, the amino acid is N-acetyl cysteine, the genistein alternative is genistein or aglycon geninstein, and the icariin alternative is icariin. More preferably, the antioxidant composition may further include resveratrol. Further preferably, the antioxidant composition may include lutein or vitamin A in addition to resveratrol. Optimally, the antioxidant composition may include one or more ingredients selected from the group consisting of resveratrol, lutein, vitamin A, vitamin E, vitamin C, selenium, alpha lipoic acid and combinations thereof. Alternatively, the antioxidant composition may include horny goat weed extract to provide icariins, d-alpha topopheryl succinate as vitamin E, calcium ascorbate as the source of vitamin C, L-selenomethionine as the source of selenium, beta carotene as the source of vitamin A, and polygnonium cuspidatum as the source of resveratrol.

In a preferred embodiment of the present invention, the antioxidant composition may be provided to a mammal in any dosage forms for the mammal to ingest or intake. Preferably, such dosage forms may include, but not limited to capsule, tablet, pill, powder and granule forms. Alternatively, the dosage may be a liquid dosage forms including, but not limited to, emulsions, suspension and spray. Preferably, exemplary one dosage of the antioxidant composition may be provided in one pill, one tablet, one teaspoon of powder or granule. Optimally, one dosage of the antioxidant composition may be provided in one capsule. It should be understood that when various dosage forms are utilized to provide the antioxidant composition to a mammal, with a preferred daily dosage, the amounts of the active ingredients of the supplement composition taken on a daily basis are the same, while the weight percentages of the ingredients may vary from dosage form to dosage form. Depending on different dosage forms, the antioxidant composition may further include a filler, binder, agent or diluter inactive substances selected from the group consisting of microcrystalline cellulose, silicon dioxide, magnesium stearate, caramel and combinations thereof.

Example 1 illustrates exemplary compositions of the present invention, which includes variations of the formulations. In a preferred embodiment, the antioxidant composition is formulated in the form of capsule and administered to a mammal in a daily dosage. Preferably, the daily dosage may be from about 1 capsule to about 10 capsules. More preferably, the daily dosage may be from about 2 capsules to about 8 capsules. Optimally, the daily dosage may be from about 4 to about 6 capsules. In a preferred embodiment, one capsule of the antioxidant composition may include from about 5 mg to about 200 mg of N-acetyl-cysteine, from about 5 mg to about 800 mg of genistein, and from about 1 mg to about 150 mg of icariin. More preferably, one capsule of the antioxidant composition may include from about 25 mg to about 100 mg of N-acetyl-cysteine, from about 25 mg to about 500 mg of genistein, and from about 5 mg to about 100 mg of icariin. Optimally, one capsule of the antioxidant composition may include about 25 mg of N-acetyl-cysteine, about 75 mg of genistein, and about 10 mg of icariin. Alternatively, the icariins is about 50 mg of horny goat weed extract, which provides for about 20% of icariin by weight.

Preferably, one capsule of the antioxidant composition may further include from about 5 mg to about 200 mg of resveratrol. More preferably, one capsule of the antioxidant composition may further include from about 10 mg to about 100 mg of resveratrol. Optimally, one capsule of the antioxidant composition may further include about 12.5 mg of resveratrol, or about 12.5 mg of polygonium cuspidatum.

Preferably, one capsule of the antioxidant composition may further include from about 0.5 mg to about 10 mg of lutein. More preferably, one capsule of the antioxidant composition may further include from about 1 mg to about 5 mg of lutein. Optimally, one capsule of the antioxidant composition may further include about 1.25 mg of lutein.

Preferably, one capsule of the antioxidant composition may further include from about 250 IU to about 5000 IU of vitamin A. More preferably, one capsule of the antioxidant composition may further include from about 500 IU to about 2500 IU of vitamin A. Optimally, one capsule of the antioxidant composition may further include about 500 IU of vitamin A, or about 500 IU of beta carotene.

Preferably, one capsule of the antioxidant composition may further include from about 30 IU to about 1500 IU of vitamin E. More preferably, one capsule of the antioxidant composition may further include from about 60 IU to about 600 IU of vitamin E. Optimally, one capsule of the antioxidant composition may further include about 30 IU of vitamin E, or about 30 IU of d-alpha topopheryl succinate.

Preferably, one capsule of the antioxidant composition may further include from about 25 mg to about 1000 mg of vitamin C. More preferably, one capsule of the antioxidant composition may further include from about 50 mg to about 500 mg of vitamin C. Optimally, one capsule of the antioxidant composition may further include about 62.5 mg of vitamin C, or about 62.5 mg of calcium ascorbate.

Preferably, one capsule of the antioxidant composition may further include from about 10 micrograms to about 100 micrograms of selenium. More preferably, one capsule of the antioxidant composition may further include from about 25 micrograms to about 50 micrograms of selenium. Optimally, one capsule of the antioxidant composition may further include about 25 micrograms of selenium, or about 25 micrograms of L-selenomethionine.

Preferably, one capsule of the antioxidant composition may further include from about 2 mg to about 200 mg of alpha-lipoic acid. More preferably, one capsule of the antioxidant composition may further include from about 5 mg to about 100 mg of alpha-lipoic acid. Optimally, one capsule of the antioxidant composition may further include about 5 mg of alpha-lipoic acid.

In a preferred embodiment, a daily dosage of four capsules of the antioxidant composition may include from about 50 mg to about 1000 mg of N-acetyl-cysteine, from about 25 mg to about 2000 mg of genistein, and from about 5 mg to about 100 mg of icariin. More preferably, four capsules of the antioxidant composition may include from about 100 mg to about 500 mg of N-acetyl-cysteine, from about 50 mg to about 1000 mg of genistein, and from about 10 mg to about 200 mg of icariin. Optimally, four capsules of the antioxidant composition may include about 100 mg of N-acetyl-cysteine, about 300 mg of genistein, and about 40 mg of icariin. Alternatively, the icariins is 200 mg of horny goat weed extract, which provides about 20% of icariin by weight.

Preferably, the daily dosage of four capsules of the antioxidant composition may further include from about 25 mg to about 1000 mg of resveratrol. More preferably, four capsules of the antioxidant composition may further include from about 50 mg to about 500 mg of resveratrol. Optimally, four capsules of the antioxidant composition may further include about 50 mg of resveratrol, or about 50 mg of polygonium cuspidatum.

Preferably, the daily dosage of four capsules of the antioxidant composition may further include from about 2.5 mg to about 50 mg of lutein. More preferably, four capsules of the antioxidant composition may further include from about 5 mg to about 25 mg of lutein. Optimally, four capsules of the antioxidant composition may further include about 5 mg of lutein.

Preferably, the daily dosage of four capsules of the antioxidant composition may further include from about 250 IU to about 10000 IU of vitamin A. More preferably, four capsules of the antioxidant composition may further include from about 500 IU to about 5000 IU of vitamin A. Optimally, four capsules of the antioxidant composition may further include about 2000 IU of vitamin A, or about 2000 IU of beta carotene.

Preferably, the daily dosage of four capsules of the antioxidant composition may further include from about 60 IU to about 2000 IU of vitamin E. More preferably, four capsules of the antioxidant composition may further include from about 120 IU to about 1000 IU of vitamin E. Optimally, four capsules of the antioxidant composition may further include about 120 IU of vitamin E, or about 120 IU of d-alpha topopheryl succinate.

Preferably, the daily dosage of four capsules of the antioxidant composition may further include from about 50 mg to about 2000 mg of vitamin C. More preferably, four capsules of the antioxidant composition may further include from about 100 mg to about 1000 mg of vitamin C. Optimally, four capsules of the antioxidant composition may further include about 250 mg of vitamin C, or about 250 mg of calcium ascorbate.

Preferably, the daily dosage of four capsules of the antioxidant composition may further include from about 20 micrograms to about 400 micrograms of selenium. More preferably, four capsules of the antioxidant composition may further include from about 50 micrograms to about 200 micrograms of selenium. Optimally, four capsules of the antioxidant composition may further include about 100 micrograms of selenium, or about100 micrograms of L-selenomethionine.

Preferably, the daily dosage of our capsules of the antioxidant composition may further include from about 5 mg to about 100 mg of alpha-lipoic acid. More preferably, four capsules of the antioxidant composition may further include from about 10 mg to about 250 mg of alpha-lipoic acid. Optimally, four capsules of the antioxidant composition may further include about 25 mg of alpha-lipoic acid.

In a preferred embodiment of the method of the present invention, the antioxidant composition may be administered to a mammal to mitigate oxidative stress. The method comprises providing an antioxidant composition in a dietary supplement form, and causing the mammal to ingest or intake an antioxidant composition comprising one or more amino acids; a genistein alternative selected from the group consisting of genistein, aglycon genistine, hormone receptive modulators, phosphordiesterase (PDE) inhibitors of types 1, 2, 3 and 4, anti-neoplastic tyrosine kinase inhibitors, and combinations thereof; and an icariin alternative selected from the group consisting of icarosides 1 or 2, PDE-4 inhibitors, PDE-5 inhibitors, derivatives of the epimedium species including icarrocides I and II, and combinations thereof. Preferably, the antioxidant composition may be administered at a preferred dosage and with a preferred frequency. More preferably, the preferred dosage and the frequency of administer a preferred dosage may also be altered for the purposes of remedying or treating various degrees of oxidative stress. Optimally, the relative percentage weight of each ingredients of the antioxidant composition may also be altered to enhance the known effectives of the ingredients. The effect of the antioxidant composition may be manifested in the oxidative damage to lymphocyte DNA inflicted by various of oxidative stress, therefore, the effect of the antioxidant composition may be quantified by measuring DNA damage (p-H2AX) using various clinical samples. Alternatively, among other methods, the effect of the antioxidant composition may also be quantified by measuring the Oxygen Radical Absorbance Capacity (ORAC) level of a serum. The benefits to bone metabolism can be measured by standard bone densiometry in both man and beast.

In a preferred embodiment of the present invention, the antioxidant composition may be administered to a mammal for treating the mammal with one or more of conditions and ailments. Such conditions and ailments include, but not limited to, osteoporosis, conditions and ailment caused by exposure to radiation, exposure to water or/and air pollution, exposure to outdoor environments, and exposure to natural or artificial light. The method comprises causing the human with the ailments or conditions to ingest a composition comprising one or more amino acids; a genistein alternative selected from the group consisting of genistein, aglycon genistine, hormone receptive modulators, phosphordiesterase (PDE) inhibitors of types 1, 2, 3 and 4, anti-neoplastic tyrosine kinase inhibitors, and combinations thereof; and an icariin alternative selected from the group consisting of icarosides 1 or 2, PDE-4 inhibitors, PDE-5 inhibitors, derivatives of the epimedium species including icarrocides I and II, and combinations thereof. The antioxidant composition may be administered at a preferred dosage and with a preferred frequency. Preferably, the preferred dosage and the frequency of administer a preferred dosage may also be altered for the purposes of remedying or treating various conditions and ailments. Optimally, the relative percentage weight of each ingredients of the antioxidant composition may also be altered to enhance the known effectives of the ingredients. For example, selenium promotes the function of NAC's regulation of the production of glutathione. For another example, PDE-4 inhibitors such as Rolipram and roflumilast (Daliresp) may be included for treatment of chronic asthma, the combination of N-acetyl cysteine and icariin may be included for treatment for lung inflammation, asthmatics and Chronic Obstructive Pulmonary (COPD). For another example, icariin, both an antioxidant and a neurotrope, increasing osteoblasts in castrated animals with diminished osteoblasts, may be included to raise Bone Mineral Density (BMD) along with genistein. For yet another example, icariin, genistein and resveratrol may be included for the preservation of cognitive function of a mammal. For still yet another example, predosing of the antioxidant compositions may affect the Reactive Oxygen Species (ROS) release of ETOH break down and the diminishment of cognitive loss from some anesthetics as well as acute ETOH consumption.

In a preferred embodiment, the antioxidant composition may be administered to a mammal for treating conditions and ailments including oxidative stress due to ionizing radiation for mitigating the adverse impact of unintended x-ray damage to DNA arising from diagnostic studies such as:
i: CT Scans
ii: Mammograms
iii: Nuclear medicinal studies
iv: angiography
v: BMD studies
vi: routine x-rays

In preferred embodiment of the present invention, the antioxidant composition may be administered to a mammal for public health protection from mass radiation caused by disasters/acts of terrorism such as “Three Mile Island”, “Fukushima”, “Hiroshima”, “Nagasaki”, “Chernobyl”, potential exposure from sunken nuclear sub reactors whose hulls might be eroding, as well as the un-detonated H-Bomb lost somewhere in the world. In addition to x-ray mitigation, the antioxidant composition may also be administered to the mammal for the protection or mitigation from Gamma, Beta, Alpha, and UV radiation. For example, the antioxidant composition may be used to mitigate:
i: UV protection from driving
ii: Tanning booths
iii: Airport scanners
iv: Unintentional Radon exposure at home and work environments
v: Air/Water Pollution

The antioxidant composition may further be administered to a mammal:
i: As adjuvant co-therapy of conventional antibiotics for increasingly resistant infectious bacterial diseases. This effect is by means of increasing the bacterial biodome permeability to common antibiotics thus achieving a greater and more therapeutic effect of the infecting bacteria.
ii: As a natural enhancer of female sexual function from a phospho-dieasterase type 4 Inhibitor (Icariin) that dilates blood flow to the Clitoris.
iii: For antibiotic resistant MRSA and acne infections.
iv: To diminish chronic Asthma and Bronchitis in smokers.
v: To enhance sexual response to the PDE-4 inhibitor Rofumilast (Daliresp).
vi: To enhance fast recovery from maximal stress exercise such as sprint 8 exercise or any high intensity exercises.

In a preferred embodiment of the present invention, the relative weights of the ingredients of an antioxidant composition may be altered for the purposes of remedying or treating various conditions and ailments. The relative percentage weight of each ingredients of the antioxidant composition may also be altered to enhance the known effectives of the ingredients. Further, the preferred daily dosage and the frequency of administer a preferred dosage may also be altered for the purposes of remedying or treating various conditions and ailments. For example, the antioxidant composition may be provided as a “radiation mitigation” formulation for conditions and ailments caused by x-ray diagnosis when taken before exposure, an “osteoporosis” formulation for bone and joint health, a “city” formulation for conditions and ailments caused by environmental pollution such as air and water pollution, a “sun” formulation for conditions caused by outdoor exposure to such as natural light, intense sunlight at high altitudes, or artificial light. Furthermore, the antioxidant composition may be provided as a “veterinary” formulation for veterinary applications. For example, the antioxidant composition may be administered to spay/neutered mammals such as cats and dogs for the purposes of increasing the health and life span of the spay/neutered mammals to address the unintended physiologic consequences of sex hormonal loss arising from spaying and neutering. The use of the antioxidant composition may delay and mitigate arthropothy and arthritis in spayed/neutered mammals by maintaining bone density from the osteoblastic effects of the genistein and icariin. The use of the antioxidant composition may further increase dental and periodontal health by maintaining bone mass of alveolar and mandibular bones for reasons stated above. The use of the antioxidant composition may also promote health bladder function and diminishing bladder stress incontinence due to hormone loss. The use of the antioxidant composition may also delays the onset of canine cognitive impairment.

In a preferred embodiment, the antioxidant composition may be manufactured by compounding or otherwise mixing the ingredients and excipients. Such excipients are substances that are of little or no therapeutic value, but useful in the manufacture and compounding of the compositions. Exemplary excipients include, but not limited to, microcrystalline cellulose, silicon dioxide, magnesium stearate, caramel and combinations thereof. The antioxidant composition may be either water or lipid soluble. Preferably, the antioxidant composition may be water soluble. The antioxidant composition may also be formulated into the form of dietary and nutritional supplement. Furthermore, the antioxidant composition may also be manufactured into a kit, comprising some ingredients of the antioxidant composition formulated into one dosage form and other ingredients into one same dosage form, or one or more different dosage forms.

In a preferred embodiment, the antioxidant composition may be included in pets' food or drink supplies. Preferably, such pet food may include on a daily basis from about 50 mg to about 200 mg of the antioxidant composition per 10 lb. More preferably, such pet food may include on a daily basis from about 75 mg to about 150 mg of the antioxidant composition per 10 lb. Optimally, such pet food may include on a daily basis from about 90 mg to about 120 mg of the antioxidant composition per 10 lb. Alternatively, such pet food may include on a daily basis about 100 mg of the antioxidant composition per 10 lb. For cat food, alternatively, a 75 g pouch of such cat food may include, for providing respective potency of the antioxidant composition, about 100 mg, or about 200 mg, or about 400 mg of the antioxidant composition.

### EXAMPLE 1

In this example of the present invention, antioxidant compositions and variations thereof were formulated in a capsule form, including appropriate inactive substances such as filler, binder, agent, diluter or other excipients selected from the group consisting of microcrystalline cellulose, silicon dioxide, magnesium stearate, caramel and combinations thereof, according to standard methods known to the one of ordinary skill in the art.

DAILY DOSE = 4 capsules

| | | per 1 | | per 4 | | Range per 1 | Range per 4 |
|---|---|---|---|---|---|---|---|
| | Actives (no excipients) | capsule | | capsules | | capsule | capsules |
| 1 | N-Acetyl-Cysteine | 25.00 | mg | 100.00 | mg | 25-250 | 100-500 |
| 2 | Genestein | 75.00 | mg | 300.00 | mg | 25-500 | 50-1000 |
| 3 | Horny Goat Weed extract (20% Icarins) | 50.00 | mg | 200.00 | mg | 25-500 | 50-1000 |
| 4 | Vitamin E (d-alpha tocopheryl succinate) | 30.00 | IU | 120.00 | IU | 60-600 | 120-1000 |
| 5 | Vitamin C (calcium ascorbate) | 62.50 | mg | 250.00 | mg | 50-500 | 100-1000 |
| 6 | Selenium (L-selenomethionine) | 25.00 | mcg | 100.00 | mcg | 25-50 | 50-200 |
| 7 | Alpha Lipoic Acid | 5.00 | mg | 20.00 | mg | 5-100 | 10-250 |
| 8 | Vitamin A (beta carotene) | 500.00 | IU | 2000.00 | IU | 500-2500 | 500-5000 |
| 9 | Resveratrol (polygonium cuspidatum) | 12.50 | mg | 50.00 | mg | 10-100 | 50-500 |
| 10 | Lutein | 1.25 | mg | 5.00 | mg | 1-5 | 5-25 |

The examples are illustrative of the present invention only and are in no way to be interpreted as limiting the scope of the present invention, as defined in the claims.

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” Where the context permits, words in the above Detailed Description of the Preferred Embodiments using the singular or plural number may also include the plural or singular number respectively. The word “or” in reference to a list of two or more items, covers all of the following interpretations of the word: any of the items in the list, all of the items in the list, and any combination of the items in the list.

The above-detailed description of embodiments of the disclosure is not intended to be exhaustive or to limit the teachings to the precise form disclosed above. While specific embodiments of and examples for the disclosure are described above for illustrative purposes, various equivalent modifications are possible within the scope of the disclosure, as those skilled in the relevant art will recognize. Further any specific numbers noted herein are only examples: alternative implementations may employ differing values or ranges.

The teachings of the disclosure provided herein can be applied to other systems, not necessarily the system described above. The elements and acts of the various embodiments described above can be combined to provide further embodiments.

Any patents and applications and other references noted above, including any that may be listed in accompanying filing papers, are incorporated herein by reference in their entirety. Aspects of the disclosure can be modified, if necessary, to employ the systems, functions, and concepts of the various references described above to provide yet further embodiments of the disclosure.

These and other changes can be made to the disclosure in light of the above Detailed Description of the Preferred Embodiments. While the above description describes certain embodiments of the disclosure, and describes the best mode contemplated, no matter how detailed the above appears in text, the teachings can be practiced in many ways. Details of the system may vary considerably in its implementation details, while still being encompassed by the subject matter disclosed herein. As noted above, particular terminology used when describing certain features or aspects of the disclosure should not be taken to imply that the terminology is being redefined herein to be restricted to any specific characteristics, features or aspects of the disclosure with which that terminology is associated. In general, the terms used in the following claims should not be construed to limit the disclosures to the specific embodiments disclosed in the specification unless the above Detailed Description of the Preferred Embodiments section explicitly defines such terms. Accordingly, the actual scope of the disclosure encompasses not only the disclosed embodiments, but also all equivalent ways of practicing or implementing the disclosure under the claims.

Accordingly, although exemplary embodiments of the invention have been shown and described, it is to be understood that all the terms used herein are descriptive rather than limiting, and that many changes, modifications, and substitutions may be made by one having ordinary skill in the art without departing from the spirit and scope of the invention.

What is claimed is:

1. An antioxidant composition comprising:
one or more amino acids; a genistein alternative selected from the group consisting of genistein, aglycon genistine, hormone receptive modulators, phosphordiesterase (PDE) inhibitors of types 1, 2, 3 and 4, anti-neoplastic tyrosine kinase inhibitors, and combinations thereof; and an icariin alternative selected from the group consisting of icarosides 1 or 2, PDE-4 inhibitors, PDE-5 inhibitors, derivatives of the epimedium species including icarrocides I and II, and combinations thereof.

2. The antioxidant composition of claim 1 wherein the amino acid is N-acetyl cysteine.

3. The antioxidant composition of claim 1 wherein the genistein alternative is genistein or aglycon genistine.

4. The antioxidant composition of claim 1 wherein the icariin alternative is icariin or icarosides 1 or 2.

5. The antioxidant composition of claim 1 further comprising an ingredient selected from the group consisting of resveratrol, vitamin E, vitamin C, selenium, vitamin A, alpha-lipoic acid, lutein, inactive substances and combinations thereof.

6. The antioxidant composition of claim 5 wherein the inactive substances are selected from the group consisting of microcrystalline cellulose, silicon dioxide, magnesium stearate, caramel and combinations thereof.

7. The antioxidant composition of claim 1 wherein the antioxidant composition comprises, in one capsule, from about 25 mg to about 250 mg of the amino acid, from about 25 mg to about 500 mg of the genistein alternative, and from about 5 mg to about 100 mg of the icariin alternative.

8. The antioxidant composition of claim 5, wherein the antioxidant composition comprises, in one capsule, from about 25 mg to about 250 mg of the amino acid, from about 25 mg to about 500 mg of the genistein alternative, and from about 5 mg to about 100 mg of the icariin alternative, from about 60 IU to about 600 IU of vitamin E, from about 50 mg to about 500 mg of the vitamin C, from about 25 micrograms to about 50 micrograms of the L-selenomethionine, from about 5 mg to about 100 mg of the alpha-lipoic acid, from about 500 IU to about 2500 IU of the vitamin A, from about 10 mg to about 100 mg of the resveratrol, and from about 1 mg to about 5 mg of the lutein.

9. The antioxidant composition of claim 5, wherein the antioxidant composition comprises, in four capsules, from about 100 mg to about 500 mg of the amino acid, from about 50 mg to about 1000 mg of the genistein alternative, and from about 10 mg to about 200 mg of the icariin alternative, from about 120 IU to about 1000 IU of the vitamin E, from about 100 mg to about 1000 mg of the vitamin C, from about 50 micrograms to about 200 micrograms of the L-selenomethionine, from about 10 mg to about 250 mg of the alpha-lipoic acid, from about 500 IU to about 5000 IU of the vitamin A, from about 50 mg to about 500 mg of the resveratrol, and from about 5 mg to about 25 mg of the lutein.

10. The antioxidant composition of claim 8, wherein the icariin is provided in form of horny goat weed extract, the vitamin E is d-alpha tocopherol succinate, the vitamin C is calcium ascorbate, the selenium is L-selenomethionine, the resveratrol is polygonium cuspidatum, and the vitamin A is beta-carotene.

11. The antioxidant composition of claim 5 wherein each ingredient is dry compounded or mixed in powder or granular form.

12. A method for a mammal to mitigate oxidative stress, the method comprising:
providing an antioxidant composition in a dietary supplement form; and
causing the mammal to intake the antioxidant composition such that the antioxidant composition reduces damage to the mammal's DNA caused by the oxidative stress,
wherein the antioxidant composition comprises one or more amino acids; a genistein alternative selected from the group consisting of genistein, aglycon genistine, hormone receptive modulators, phosphordiesterase (PDE) inhibitors of types 1, 2, 3 and 4, anti-neoplastic tyrosine kinase inhibitors, and combinations thereof; and an icariin alternative selected from the group consisting of icarosides 1 or 2, PDE-4 inhibitors, PDE-5 inhibitors, derivatives of the epimedium species including icarrocides I and II, and combinations thereof.

13. The method of claim 12, wherein the antioxidant composition further comprises an ingredient selected from the group consisting of resveratrol, vitamin E, vitamin C, L-selenomethionine, vitamin A, alpha-lipoic acid, lutein, inactive substances and combinations thereof.

14. The method of claim 12 wherein the antioxidant composition comprises, in a preferable daily dosage, from about 100 mg to about 500 mg of the amino acid, from about 50 mg to about 1000 mg of the genistein alternative, and from about 10 mg to about 200 mg of the icariin alternative.

15. The method of claim 14 wherein the antioxidant composition further comprises, in a preferable daily dosage, from about 120 IU to about 1000 IU of the vitamin E, from about 100 mg to about 1000 mg of the vitamin C, from about 50 micrograms to about 200 micrograms of the L-selenomethionine, from about 10 mg to about 250 mg of the alpha-lipoic acid, from about 500 IU to about 5000 IU of the vitamin A, from about 50 mg to about 500 mg of the resveratrol, and from about 5 mg to about 25 mg of the lutein.

16. A method of treating a mammal with one or more of the following conditions and ailments including osteoporosis, exposure to radiation, gonad hormone deficiency either natural or surgical caused, exposure to water or/and air pollution, exposure to outdoor environments, and exposure to natural or artificial light, the method comprising causing the mammal with the ailments or conditions to ingest or intake a composition comprising one or more amino acids; a genistein alternative selected from the group consisting of genistein, aglycon genistine, hormone receptive modulators, phosphordiesterase (PDE) inhibitors of types 1, 2, 3 and 4, anti-neoplastic tyrosine kinase inhibitors, and combinations thereof; and an icariin alternative selected from the group consisting of icarosides 1 or 2, PDE-4 inhibitors, PDE-5 inhibitors, derivatives of the epimedium species including icarrocides I and II, and combinations thereof.

17. The method of claim 16, wherein the composition further comprises an ingredient selected from the group consisting of resveratrol, vitamin E, vitamin C, L-selenomethionine, vitamin A, alpha-lipoic acid, lutein, inactive substances and combinations thereof.

18. The method of claim 16, wherein the antioxidant composition comprises, in a preferable daily dosage, from about 100 mg to about 500 mg of the amino acid, from about 50 mg to about 1000 mg of the genistein alternative, and from about 10 mg to about 200 mg of the icariin alternative.

19. The method of claim 18, wherein the antioxidant composition further comprises, in a preferable daily dosage, from about 120 IU to about 1000 IU of the vitamin E, from about 100 mg to about 1000 mg of the vitamin C, from about 50 micrograms to about 200 micrograms of the L-selenomethionine, from about 10 mg to about 250 mg of the alpha-lipoic acid, from about 500 IU to about 5000 IU of the vitamin A, from about 50 mg to about 500 mg of the resveratrol, and from about 5 mg to about 25 mg of the lutein.
